(19) United States
(12) Patent Application Publication
(10) Pub. No.: US 2026/0207199 A1
(43) Pub. Date: Jul. 23, 2026

(54) DEVICES AND METHODS FOR END-TO-END ANASTOMOSIS

(71) Applicant: M. I. Advanced Thermosurgery, Inc., Hanover, NH (US)

(72) Inventor: Csaba TRUCKAI, Saratoga, CA (US)

(21) Appl. No.: 19/139,328

(22) PCT Filed: Dec. 14, 2023
(86) PCT No.: PCT/US2023/084107

§ 371 Date: Jun. 13, 2025

Related U.S. Application Data
(60) Provisional application No. 63/387,415, filed on Dec. 14, 2022.

(51) Int. Cl. A61B 17/11

(52) U.S. Cl. CPC A61B 17/1114; A61B 2017/1103; A61B 2017/1132

(57) ABSTRACT

Methods and devices for performing an end-to-end anastomosis system, particularly an anastomosis in a patient's colon by minimally invasive surgery. In various aspects the end-to-end anastomosis is facilitated by clamping, delivery of an array of staples via a surgical stapling device or using a tissue welding device and technique using electrodes on a clamping device.

### PRIORITY CLAIM

This patent application claims priority to U.S. provisional Ser. No. 63/387,415, titled “DEVICES AND METHODS FOR END-TO-END ANASTOMOSIS,” and filed on Dec. 14, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates to performing an end-to-end anastomosis system, particularly an anastomosis in a patient's colon, by minimally invasive surgery.

### BACKGROUND

Colorectal cancer is the third most common cancer worldwide, with over 1.9 million new cases in 2020. Colorectal cancer is the third most commonly occurring cancer and the second most commonly occurring cancer in women globally. The global incidence of colorectal cancer is expected to increase by 60% to over 2.2 million new cases and 1.1 million annual deaths by the year 2030. The causes of such cancers are certain but are likely related to obesity, processed foods, smoking, and more sedentary lifestyles.

Treatments for colorectal cancer typically include resection of a diseased part of the patient's colon. Following such a resection, a surgical anastomosis is performed wherein the free ends of the intestine are reconnected. One of the most serious complications of anastomosis is anastomotic leakage which occurs in up to 20% of patients undergoing such a resection procedure. Leakage from the anastomosis site causes fecal material to enter the abdominal cavity and can lead to serious complications such as peritonitis, septic shock and can be life threatening. Such an anastomotic leakage is not always immediately detectable and can result in re-operations and extended times in hospitals.

### SUMMARY

In one embodiment, there is a method of providing end-to-end anastomosis in an intestine of a patient. One step in the method includes resecting a portion of the intestine leaving a first free end of the intestine and a second free end of the intestine. The intestine having a wall comprising a mucosa, a muscularis, and a serosa, where the muscularis and the serosa comprise an outward layer of the intestine. Next, there is a step of separating the mucosa from the outward layer at the first free end to provide an elongated free end of the mucosa at the first free end.

There is also a step of positioning the elongated free end of the mucosa in a lumen of the second free end. In another step, there is attaching the outward layer of the first free end to a wall of the second free end at an interface to provide an end-to-end anastomosis. In one aspect, the elongated free end of the mucosa overlaps the interface to prevent intestinal leakage at the interface.

In additional aspects, prior to attaching the outward layer of the first free end to the wall of the second free end, the outward layer of the first free end and the wall of the second free end are everted or folded outwardly. Optionally, prior to attaching the outward layer of the first free end to the wall of the second free end, the outward layer of the first free end and the wall of the second free end are folded inwardly. In some aspects, the elongated free end of the mucosa has a length of at least 5 mm, at least 10 mmm, or at least 20 mm.

In some additional aspects, the elongated free end of the mucosa is positioned in the lumen of the second free end with the assistance of an intraluminal guide member. In some instances, attaching includes approximating the first free end and the second free end using a clamping device. In some instances, attaching is provided by a plurality of staples delivered from a stapler head of the clamping device. In some instances, attaching is provided by tissue welding from RF electrodes in the clamping device. In still other instances, attaching is provided by a plurality of staples in combination with tissue welding provided by the clamping device.

Optionally, approximating the first free end and the second free end comprises approximating the first free end and the second free end in a flattened position. Additionally or optionally, approximating the first free end and the second free end occurs when the first free end and the second free end are in an open-lumen position. In some instances, the intraluminal guide member is configured with suction ports for engaging the elongated free end of the mucosa.

### BRIEF DESCRIPTION OF THE DRAWINGS

FIG. 1 is a schematic view of a portion of the patient's colon and mesentery with a diseased section, for example, including a tumor.

FIG. 2 is a schematic view similar to that of FIG. 1, showing the diseased portion of the colon following resection in a method of the invention, leaving first and second free ends of the colon.

FIG. 3 is a subsequent step of the method wherein incisions are made around the muscularis and serosa of the first free end in preparation of dissecting the outer layers away from the mucosa.

FIG. 4 is another step of the method wherein the first free end of the colon's outer layers are everted over the non-everted mucosa at the same time the second free end of the colon is everted.

FIG. 5 illustrates a subsequent step of the method wherein clamping heads of a stapler are inserted into opposing spaces underlying the everted sections of the colon while, at the same time, a guide device is introduced intraluminal in the colon.

FIG. 6 is an enlarged sectional view of the first and second free ends of the colon, as shown in FIG. 5, wherein the mucosa of the first free end is disposed in the lumen of the second free end.

FIG. 7 shows a subsequent step of the method wherein the clamping heads are moved together to clamp together the first and second free ends.

FIG. 8 illustrates the connection of the first and second free ends following stapling removal of the stapling device, again showing the free end of the mucosa extending distally into the lumen of the second free end of the colon wherein the mucosal overlap assists in preventing any leakage from the intestine through the stapled connection.

FIG. 9 shows another variation similar to that of FIG. 8, where the first and second free ends are clamped together and connected with staples but without substantial lengths of everted free ends of the colon together with an intraluminal guide.

FIG. 10A shows another variation of a method of end-to-end anastomosis using an intraluminal circular stapler together with an elongated dissected mucosal layer for overlapping the connection.

FIG. 10B shows another step of the method of FIG. 10A following removal of the intraluminal stapler wherein the elongated free end of the mucosa overlaps the internally stapled ends of the colon segments.

FIG. 11 shows another variation of a method of end-to-end anastomosis wherein the colon segments are thermally welded together by use of RF electrodes together with an elongated dissected mucosal layer for overlapping the connection.

### DETAILED DESCRIPTION

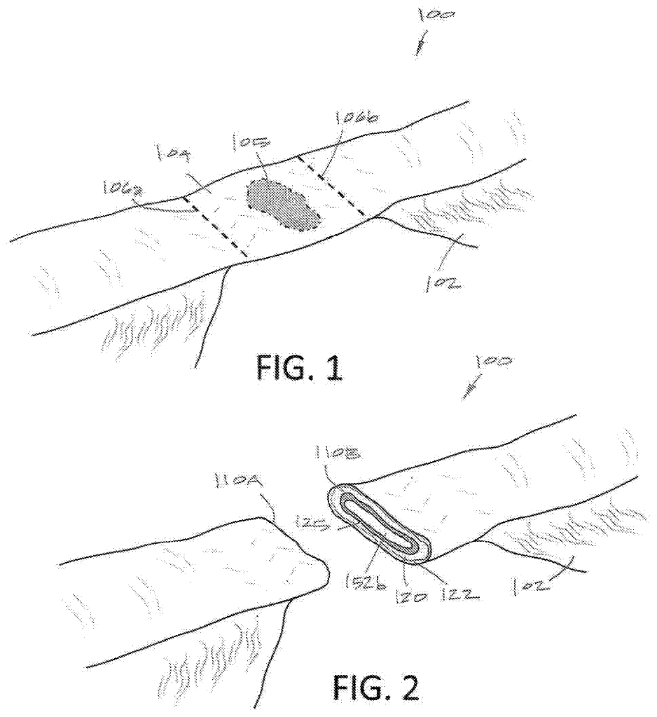

Referring to the drawings, and initially to FIGS. 1 to 8, a method of the invention is illustrated for an end-to-end anastomosis in a patient's colon following a resection procedure.

FIG. 1 is a schematic view of a portion of the patient's colon 100 and mesentery 102 with a diseased section 104 of the colon having a cancerous growth or tumor 105. Dashed lines 106a and 106b are shown where the diseased section 104 can be resected. FIG. 2 illustrates a subsequent step of the method following resection of the diseased portion 104 of the colon 100 leaving a first, healthy free end 110A of the colon and a second healthy, free end 110B of the remaining colon.

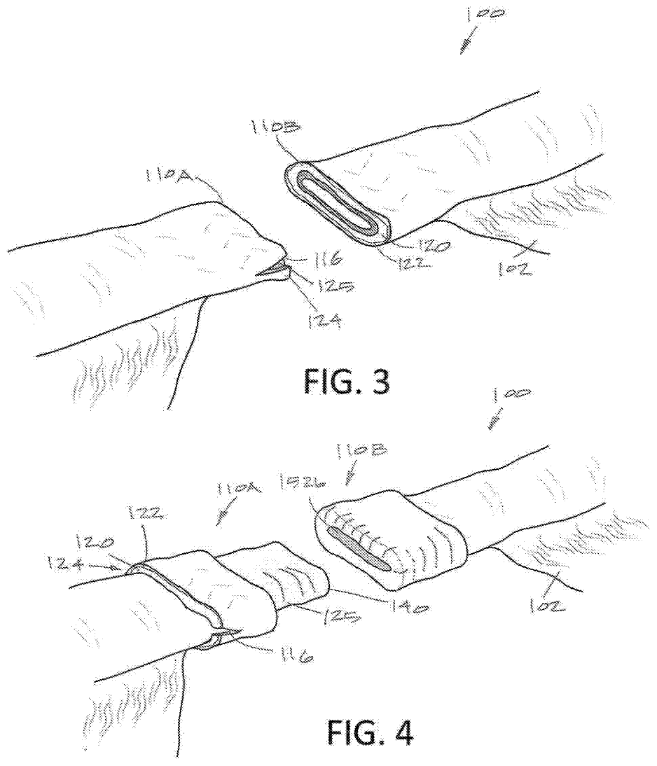

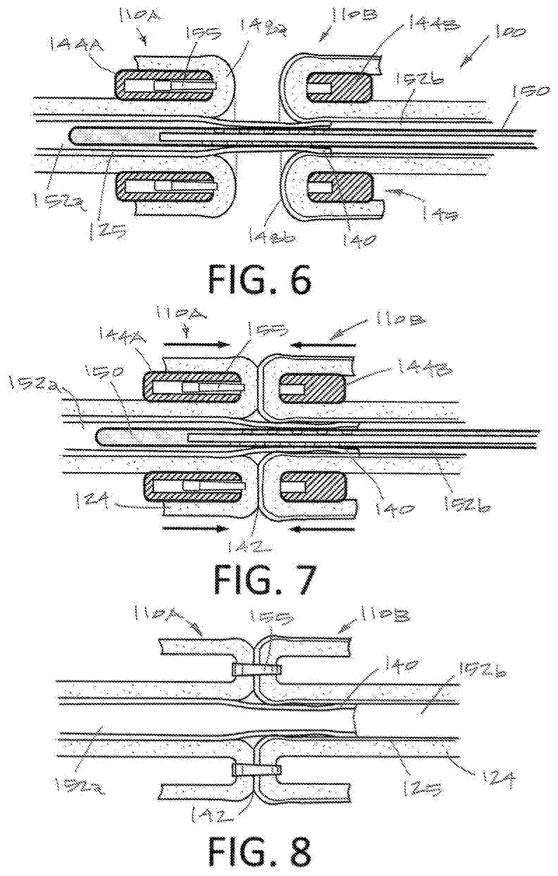

FIG. 3 illustrates a subsequent optional step of the method wherein one or more incisions 116 are made around the muscularis 120 and serosa 122 of the first free end 110A to assist in dissecting the outer layers 124 (i.e., muscularis 120 and serosa 122) away from the mucosa 125. Various tools can be used to grasp the mucosa 125 and to grasp the outer layers 124 to thereafter evert the outer layers. FIG. 4 then shows the muscularis 120 and serosa 122 of the first free end 110A of the colon everted over the non-everted mucosa 125. The mucosa 125 then has an elongated mucosal free end 140 that can be used to overlap the interface 142 (FIG. 6) between the attached colon segments as will be described below. FIG. 4 also shows the second free end 110B of the colon in an everted configuration without the mucosa 125 being dissected away from the outer layers 124.

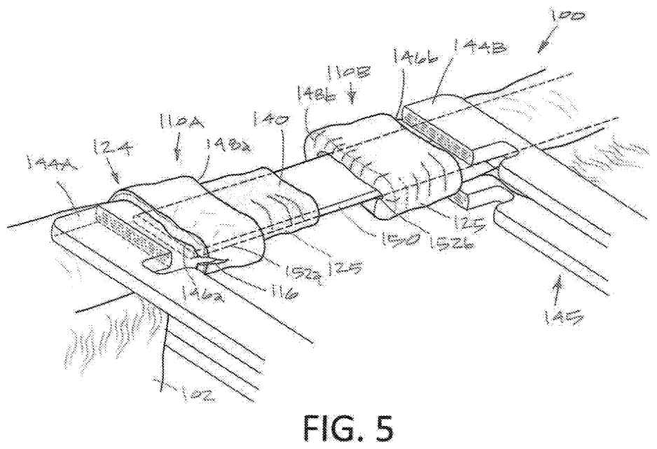

FIG. 5 next illustrates a subsequent step of the method wherein first and second clamping heads 144A, 144B of a stapler 145 are inserted into opposing spaces 146a, 146b underlying the everted portions and creases 148a, 148b of the free ends 110A and 110B. At the same time, a guide device 150 is shown after being introduced through the lumen 152b of the second free end 110A the lumen 152a of the first free end 110B. Such a guide device 150 is introduced from the exterior of the patient into the patient's colon. The anastomosis is configured obviously so that the mucosal free end 140 extends downstream in the patient's colon.

FIG. 6 is a sectional view of the first and second free ends 110A, 110B of the colon 100 as shown in FIG. 5, wherein the mucosal free end 140 of the first free end 110A is disposed in the lumen 152b of the second free end 110B. In this this variation, it can be seen that the first clamping head 144A carries staples 155 that can be actuated by a typical mechanism as is known in the art, and the second clamping head 144B comprises an anvil for deflecting and bending the staples 155 to connect the free ends 110A, 110B as is known in the art.

FIG. 7 shows a subsequent step wherein the first and second clamping heads 144A, 144B have been moved toward one another to approximate or clamp together the first and second free ends 110A, 110B of the colon 100. In FIG. 7, it can be seen that the elongated free end 140 of the mucosa 125 extends distally into the lumen 152b of the second free end 110B.

FIG. 8 then illustrates the connection of the first and second free ends 110A, 110B of the colon 100 following stapling. FIG. 8 also shows the removal of the clamping heads 144A, 144B to complete the end-to-end anastomosis procedure. As can be seen in FIG. 8, the mucosal free end 140 extends distally into the lumen 152b of the second free end 110B and overlaps the interface 142 of the free ends 110A, 110B and assists in preventing any leakage from the intestine through the stapled interface 142. It is believed that the overlapping portion 158 of the mucosal free end 140 will attach and resurface that section of the connected lumen within a matter of days. The length of the dissected mucosal free end 220 preferably extends at least 5 mm, or at least 10 mm, or at least 20 mm over and past the interface 142 between the attached colon segments.

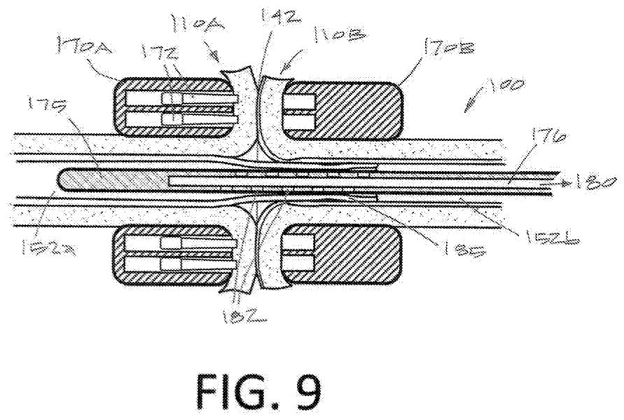

FIG. 9 shows another variation of an end-to-end anastomosis method and a stapling device similar to the method of FIGS. 6-8. In FIG. 9, the first and second free ends 110A, 110B of the colon 100 are clamped together and connected with stapler heads 170A and 170B that carry two rows of staples 172. In this variation, the first and second free ends 110A, 110B of the colon 100 are not everted over any substantial length compared to FIGS. 6-8. The intraluminal guide 175 has an aspiration channel 176, therein communicating with a negative pressure source 180. Suction ports 182 in the guide allow for the free end 185 of the mucosa to be suctioned into engagement with the guide 175 and then moved and positioned in the lumen 152b of the second free end 110B. Thereafter, graspers or other tools can be used to fold outwardly the walls of the first and second free ends 110A, 110B for engagement and stapling by the stapler heads 170A and 170B. In other aspects, the variation of FIG. 9 is similar to the method of FIGS. 6-8.

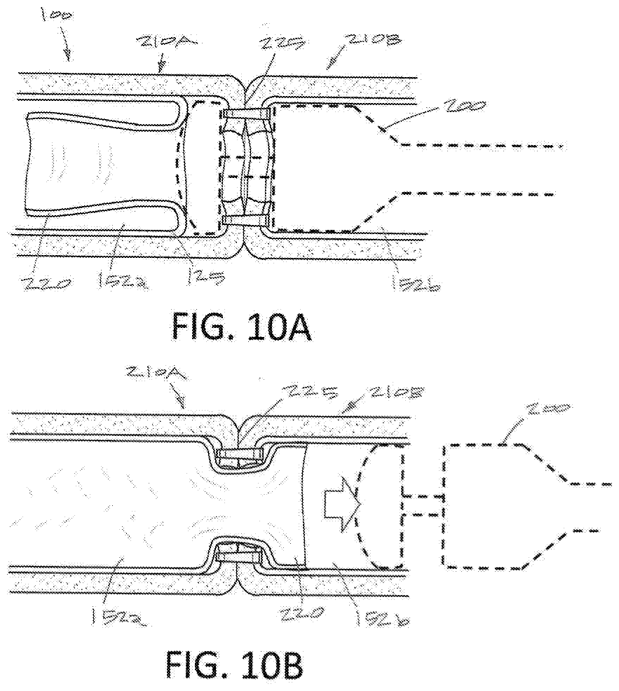

FIGS. 10A-10B show another variation of a method of end-to-end anastomosis that again provided for an elongated dissected mucosal layer that is adapted to overlap the stapled interface. However, in this variation, a conventional type of intraluminal circular stapler 200 is used. In this variation, the first and second free ends 210A, 210B of the colon 100 are folded inward, as known in the use of such a circular stapler 200. FIG. 10A shows the free ends 210A, and 210B folded inwardly and stapled together after a circular cutter of the stapler 200 has resected a central portion of the free ends 210A and 210B. In FIG. 10A, it can be seen that the elongated, dissected free end 220 the mucosa 125, has been inverted in the proximal direction into the lumen 152a of the first free end 210A of the colon before the use of the circular stapler 200. FIG. 10B then shows the withdrawal of the circular stapler 200 and the unfolding de-inverting of the free end 220 of the mucosa in the distal direction to overlap the interface 225 between the connected colon free ends 210A and 210B. Thus, this variation also provides for the rapid regrowth of the mucosa 125 over the connection interface 225, which again can prevent leakage from the intestinal lumen. Again, the length of the dissected mucosa-free end 220 preferably extends at least 5 mm, or at least 10 mm or at least 20 mm over and past the connection interface 225 between the free ends 210A and 210B.

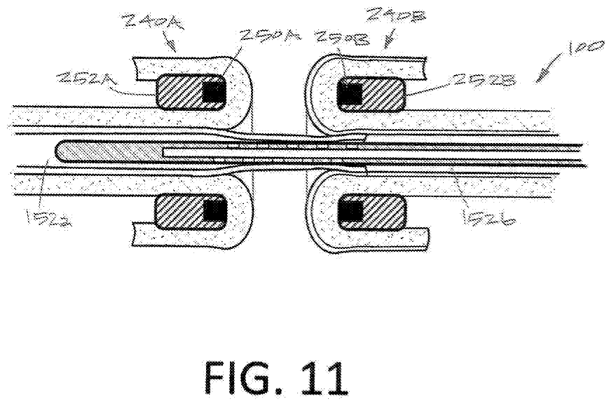

FIG. 11 illustrates another variation of a method of end-to-end anastomosis that again provides the elongated mucosal layer that overlaps an interface between connected colon segments 240A, 240B of the colon 100. In this variation, the free ends of the colon segments 240A, 240B are thermally welded together by means of opposing polarity RF electrodes 250A and 250B in the respective clamp members 252A and 252B. The use of RF electrodes and thermal welded connections in intestinal anastomosis is known in the art, for example, as disclosed in U.S. Pat. No. 8,303,610.

It should be appreciated that the inventive aspect of the invention, which comprises providing a dissected mucosal layer positioned to overlap the interface between connected colon segments, includes the use of any connecting or attachment mechanisms, including sutures, staples, thermal welding, magnetic collars or elements, spiked connection collars whether or not bio-erodible, adhesives or any combination thereof. Such connection collars are disclosed in U.S. Pat. Nos. 4,917,090; 5,250,058; 6,524,322, and applications WO2022/171349 and WO2020/225,603.

In addition, the method of everting the colon segments includes the use of everting devices of the types shown in U.S. Patents and applications U.S. Pat. Nos. 6,562,053; 6,575,985; 3,057,355 and U.S. 2005/0043749.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

All references, including publications, patent applications, and patents, cited herein are hereby incorporated by reference to the same extent as if each reference were individually and specifically indicated to be incorporated by reference and were set forth in its entirety herein.

What is claimed is:

1. A method of providing end-to-end anastomosis in an intestine of a patient, comprising:
resecting a portion of the intestine leaving a first free end of the intestine and a second free end of the intestine, the intestine having a wall comprising a mucosa, a muscularis, and a serosa, where the muscularis and the serosa comprise an outward layer of the intestine;
separating the mucosa from the outward layer at the first free end to provide an elongated free end of the mucosa at the first free end;
positioning the elongated free end of the mucosa in a lumen of the second free end; and
attaching the outward layer of the first free end to a wall of the second free end at an interface to provide an end-to-end anastomosis;
wherein the elongated free end of the mucosa overlaps the interface to prevent intestinal leakage at the interface.

2. The method of claim 1 wherein prior to attaching the outward layer of the first free end to the wall of the second free end, the outward layer of the first free end and the wall of the second free end are everted or folded outwardly.

3. The method of claim 1 wherein prior to attaching the outward layer of the first free end to the wall of the second free end, the outward layer of the first free end and the wall of the second free end are folded inwardly.

4. The method of claim 1 wherein the elongated free end of the mucosa has a length of at least 5 mm, at least 10 mmm, or at least 20 mm.

5. The method of claim 1 wherein the elongated free end of the mucosa is positioned in the lumen of the second free end with the assistance of an intraluminal guide member.

6. The method of claim 1 wherein attaching comprises approximating the first free end and the second free end using a clamping device.

7. The method of claim 6 wherein attaching is provided by a plurality of staples delivered from a stapler head of the clamping device.

8. The method of claim 6 wherein attaching is provided by tissue welding from RF electrodes in the clamping device.

9. The method of claim 6 wherein attaching is provided by a plurality of staples in combination with tissue welding provided by the clamping device.

10. The method of claim 9 wherein approximating the first free end and the second free end comprises approximating the first free end and the second free end in a flattened position.

11. The method of claim 9 wherein approximating the first free end and the second free end occurs when the first free end and the second free end are in an open-lumen position.

12. The method of claim 5 wherein the intraluminal guide member is configured with suction ports for engaging the elongated free end of the mucosa.
